(19) United States
(12) Patent Application Publication
(10) Pub. No.: US 2024/0165041 A1
(43) Pub. Date: May 23, 2024

(54) Softgel Capsule and Method of Marking a Softgel Capsule

(71) Applicant: R.P. Scherer Technologies, LLC, Carson City, NV (US)

(72) Inventors: Michael Seip, Erbach (DE); German Hamm, Erbach (DE); Frank Tewes, Eberbach (DE); Vicki Dey-Weisbecker, Heilbronn (DE); Sonja Heigenhauser, Ingelheim am Rhein (DE); Michael Schmidt, Ingelheim am Rhein (DE); Manuela Meusel, Ingelheim am Rhein (DE); Jochen Scher, Ingelheim am Rhein (DE)

(21) Appl. No.: 18/516,302

(22) Filed: Nov. 21, 2023

Related U.S. Application Data
(60) Provisional application No. 63/427,536, filed on Nov. 23, 2022.

(51) Int. Cl. A61K 9/48

(52) U.S. Cl. CPC A61K 9/4883; A61K 9/4816; A61K 9/485

(57) ABSTRACT

A softgel capsule is provided including a fill material and a shell composition The shell composition includes a marking formulation including a marking component. The marking component includes a metal oxide. Laser irradiation may be applied to the softgel capsule at a wavelength of about 200 nm to about 400 nm, causing the marking component to change color. A method of marking and marking formulation is also provided.

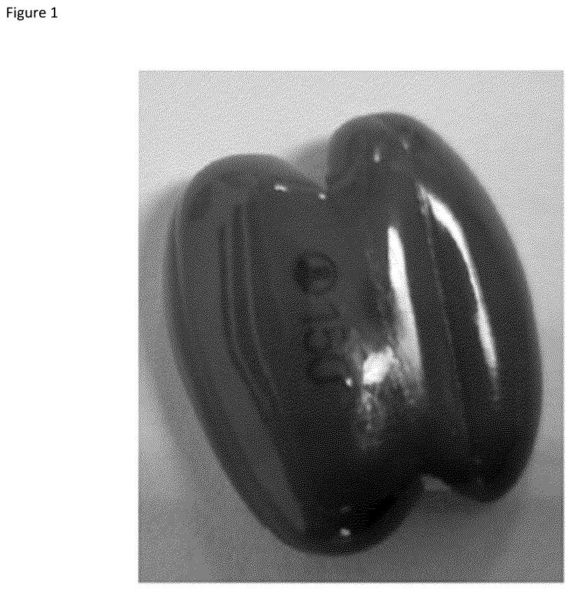

### CROSS REFERENCE TO RELATED APPLICATION(S)

The present applications claims priority to U.S. Provisional Patent Application No. 63/427,536 filed on Nov. 23, 2022, the entire contents of which is incorporated by reference in its entirety.

### TECHNICAL FIELD OF THE INVENTION

The present invention relates to a softgel capsule for encapsulating a fill composition including a shell composition, wherein the shell composition includes a marking formulation including a marking component. More specifically, the present invention relates to applying laser irradiation to a softgel capsule at a wavelength of about 100 nm to about 400 nm, causing the marking component to change color.

### BACKGROUND OF THE INVENTION

In the pharmaceutical industry, it is good manufacturing practice to mark capsules to determine the type of product, the dose, the manufacturer, and other identifying information. There are several methods that can be used to mark both hard and soft capsules. The methods are used to mark the capsule skin and/or shell of the capsule.

Some of these methods are ink printing, a carbon dioxide (CO₂) laser or a UV laser. During the ink printing method, ink may be placed on the capsule skin by different methods, such as ink jet, offset, or pad printing. A variety of colors may be used in the ink printing method. The ink printing method has its disadvantages. For example, this method touches the surface of the capsule. Because ink is soluble with certain solvents, it can be wiped off. The ink can also be wiped off if the solvent within the capsule migrates to the capsule skin surface.

During the CO₂ laser method, a CO₂ laser is used to engrave into the capsule skin. As it engraves, a white marking appears in the capsule skin or shell. This is believed to be because of the reflective changes of the engraved material. A disadvantage of this method is that oil on a capsule surface may fill and flatten the engraved surface. Thus, this may cause the visibility of the engraving to vanish.

Another common method to mark a capsule is using a UV laser. Typically, titanium oxide, such as titanium dioxide (TiO₂), is added to the capsule material, such that it reacts with the UV laser by changing color. Often times, the reaction of the TiO₂ causes the marking to appear dark on the capsule. Recently, TiO₂ has been discouraged from being used in the industry. Therefore, a need exists in the art for an alternative marking component to be used with a UV laser.

### OBJECTS AND SUMMARY OF THE INVENTION

In an embodiment of the present invention, a softgel capsule is provided. The softgel capsule includes a fill material, and a shell composition. The shell composition may include a marking formulation including a marking component. Laser irradiation may be applied to the softgel capsule of the present disclosure at a wavelength of about 100 nm to about 400 nm, causing the marking component to change color and/or change in appearance.

In an embodiment of the softgel capsule, the marking component may include a metal oxide. In some embodiments, the metal oxide may include iron oxide. The iron oxide may include iron oxide red, iron oxide yellow, iron oxide brown, iron oxide black or a combination thereof.

In some embodiments, the marking formulation may further include a plasticizer and gelatin. In some embodiments, the marking formulation may be substantially free of titanium dioxide. In some embodiments, the marking formulation does not include titanium dioxide.

In some embodiments, the laser irradiation may be applied to the softgel capsule at a wavelength of about 300 nm to about 375 nm. In an embodiment, the laser irradiation may be achieved using a UV laser.

In another embodiment of the present disclosure, a method for marking a softgel capsule is provided. The method may include irradiating a softgel capsule with a laser, wherein the softgel capsule includes a shell composition and a fill material, wherein the shell composition may include a marking formulation having a marking component.

In some embodiments of the method, the irradiating may be performed using a wavelength below about 400 nm. In some embodiments, the irradiating may be performed using a wavelength of about 100 nm, about 150 nm to about 400 nm, about 200 nm to about 350 nm, or about 250 nm to about 300 nm. In some embodiments, the irradiating may be performed using a UV laser.

In some embodiments of the method, after irradiating the softgel capsule, the marking component undergoes a color change and/or a change in appearance in the shell.

In another embodiment of the present disclosure, a marking formulation is provided. The marking formulation may include a marking component including a metal oxide, a plasticizer, and gelatin. Laser irradiation may be applied to the marking formulation at a wavelength of about 100 nm to about 400 nm, causing the marking component to change color and/or appearance.

In an embodiment of the marking formulation, the metal oxide may include iron oxide. In some embodiments, the iron oxide may include iron oxide red, iron oxide yellow, iron oxide brown, iron oxide black, iron oxide blue, iron oxide green, or a combination thereof.

In some embodiments of the marking formulation, the marking component may be included in an amount of about 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, about 1 wt % to about 4 wt %, about 1.5 wt % to about 3 wt %, or about 2 wt % to about 2.5 wt %, based on a total weight of the marking formulation.

In some embodiments of the marking formulation, the metal oxide may be included in an amount of about 1 gram to about 5 grams, about 1.2 grams to about 4.5 grams, about 1.4 grams to about 4 grams, about 1.6 grams to about 3.5 grams, about 1.8 grams to about 3 grams, or about 2 grams to about 2.5 grams, based on 100 grams of total weight of the marking formulation.

In some embodiments of the marking formulation, the plasticizer may be included in an amount of about 5 wt % to about 40 wt %, about 10 wt % to about 35 wt %, about 15 wt % to about 30 wt %, or about 20 wt % to about 25 wt %, based on a total weight of the marking formulation.

In some embodiments of the marking formulation, the gelatin may be included in an amount of about 20 wt % to about 70 wt %, about 25 wt % to about 65 wt %, about 30 wt % to about 60 wt %, about 35 wt % to about 55 wt %, or about 40 wt % to about 50 wt %, based on a total weight of the marking formulation.

In some embodiments, the gelatin may be Type A gelatin, Type B gelatin, a hide gelatin, a bone gelatin, or a combination thereof. In some embodiments, the gelatin may be 150 bloom gelatin, 200 bloom gelatin, 250 bloom gelatin, or a combination thereof.

In some embodiments, the plasticizer may be glycerol, glycerin, sorbitol, sorbitol sorbitan solution, triacetin, polysorbate, or combinations thereof.

In some embodiments, the marking formulation is substantially free of titanium dioxide. In other embodiments, the marking formulation does not include titanium dioxide. In some embodiments, the marking formulation may further include water.

### BRIEF DESCRIPTION OF THE DRAWINGS

The disclosure described herein is illustrated by way of example and not by way of limitation in the accompany FIGURE.

The FIGURE represents the results of a laser irradiation test of the Example.

### DETAILED DESCRIPTION

The detailed description set forth below is intended to merely as a description of the presently preferred embodiments of the invention, and is not intended to represent the only form in which the present invention may constructed or utilized. The description sets forth the functions, means, and methods of implementing the invention in connection with the illustrated embodiments. It is to be understood, however, that the same or equivalent functions and features may be accomplished by different embodiments that are also intended to be encompassed within the spirit and scope of the claims.

As used herein, the singular forms “a,” “an,” and “the” include plural references unless the context clearly indicates otherwise. Thus, for example, reference to “an active pharmaceutical ingredient” includes a single active pharmaceutical ingredient as well as a mixture of two or more different active pharmaceutical ingredients, and reference to an “excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±5%, such that “about 10” would include from 9.5 to 10.5.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

As used herein, the terms “active agent,” “active ingredient,” “active pharmaceutical ingredient,” “API,” and “drug” refer to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, “shell” or “shell composition” refers to the shell of a softgel capsule which encapsulates a fill material.

As used herein, “fill material” or “fill” refers to the composition that is encapsulated by the shell.

As used herein, the term “color change” or “change color” refers to a component that may change appearance including, but not limited to black, white, or grey.

As used herein, “free or substantially free” refers to a composition that comprises less than about 1 wt %, less than about 0.5 wt %, less than about 0.25 wt %, less than about 0.1 wt %, less than about 0.05 wt %, less than about 0.01 wt %, or 0 wt % of the component.

In certain embodiments, the present invention is directed to a softgel capsule that may include a fill material and a shell composition. The shell composition may include a marking component, wherein laser irradiation is applied to the softgel capsule at a wavelength of about 100 nm to about 400 nm causing the marking component to change color and/or appearance in the shell composition. In certain embodiments, it has been found that when laser irradiation is applied to the capsule, the marking component, e.g., the iron oxide, changes to a darker pigmentation, such as black.

In some embodiments, the marking component may include a metal oxide. In certain embodiments of the softgel capsule, the metal oxide may include an iron oxide. The iron oxide may be an iron oxide red, iron oxide yellow, iron oxide black, iron oxide brown, iron oxide blue, iron oxide green, or a combination thereof. In certain embodiments of the softgel capsule, the marking component does not include titanium dioxide. In other embodiments of the softgel capsule, the marking component is substantially free of titanium dioxide.

In some embodiments, the shell composition may further include a plasticizer and gelatin.

In some embodiments of the softgel capsule, the laser irradiation is achieved by using a UV laser. Without being limited to a theory, the inventors believe that the UV laser reacts with the marking component in the shell composition and causes a color change and/or a change in appearance through the light of the UV laser. In some embodiments, the color change may include a darkening of the marking component. In some embodiments, the laser irradiation may be applied to the softgel capsule at a wavelength of about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, or about 400 nm, or any value therein.

In some embodiments, the shell may include a film forming polymer and optionally a plasticizer. The film forming polymer may be an animal derived polymer, non-animal derived polymer, or a combination thereof. The animal derived polymer may include gelatin. The gelatin in the shell composition may include, but is not limited to, Type A gelatin, Type B gelatin, a hide gelatin, a fish gelatin, porcine gelatin and/or a bone gelatin used alone or in combination. In an embodiment, the gelatin is Type A medium to high Bloom gelatin. In an embodiment, the gelatin is Type B medium to high Bloom gelatin. Medium bloom is when the bloom is from about 70 grams to about 160 grams. High bloom is when the bloom is about 175 grams or above, or from about 175 grams to about 300 grams. In one embodiment, the gelatin is a 250 bloom gelatin. In another embodiment, there is only one type of gelatin. In yet another embodiment, the gelatin is a combination of at least two types of gelatins. The non-animal derived polymer may include carrageenan.

The plasticizer of the shell composition may be glycerol, glycerin, sorbitol, sorbitol sorbitan solution, a polyethylene sorbitan monooleate or a combination thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

The shell may also include additional components. The additional components may include dextrose, pectin, a gelling agent, a thickening agent, buffer, water, or a combination thereof. The shell may further include pullulan.

In some embodiments, the shell composition may include pectin. In some embodiments, the pectin may be a low methoxy pectin. In some embodiments, the pectin may be an amidated pectin, non-amidated pectin or a combination thereof. In an embodiment, the pectin is low methylester (LM) pectin with Degree of Esterification lower than 50. In some embodiments, the pectin is LMS-318, SPL-12, LM-102 AS-Z and/or LM-12 CG-Z. In other embodiments, the low methoxy (LM) pectin may be LM Pectin (P-25), LM Pectin (445C), LM Pectin (100C) or a combination thereof.

In some embodiments, the shell composition may also include a gelling agent. In some embodiments, the gelling agent may be a gellan gum. For example, the gellan gum may be a Kelcogel CG-LA gellan gum. In other embodiments, the gelling agent may be a non-animal derived gelling agent. The non-animal derived gelling agent may include carrageenan, starch, xanthan gum, agar, pectin, sugar, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcellaran, egg white powder, lacto albumin, soy protein, chitosan or a combination thereof.

In some embodiments, the shell composition may optionally include a synthetic polymer. The synthetic polymer may be polyvinyl alcohol, polyvinyl alcohol-polyethylene glycol graft co-polymer, high molecular weight polyethylene glycol, povidone, a surfactant, a nonionic triblock copolymer, or a combination thereof. In some embodiments, the synthetic polymer may be a nonionic triblock copolymer. In some embodiments, the nonionic triblock copolymer may include polyethylene oxide and polypropylene oxide blocks. In some embodiments, the surfactant may be sodium lauryl sulfate.

In some embodiments, the shell composition may include a buffer. The buffer may be dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate, dibasic potassium phosphate and a combination thereof.

In some embodiments, the shell composition may optionally comprise additional agents such as coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein. It is to be understood by one of skill in the art that the coloring agent of the shell may be separate and distinct from that of the marking component.

Exemplary suitable flavoring agents may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In some embodiments of the softgel capsule, a methacrylic copolymer may be included in the shell composition. In some embodiments, the methacrylic copolymer may be Kollicoat MAE 100P.

The thickening agent may be a starch, a starch derivative or a modified starch. The starch or starch derivative may be hydroxypropylated tapioca starch, hydroxypropylated corn starch, potato starch, or pregelatinized modified corn starches. The modified starch includes such starches as hydroxypropylated starches, acid thinned starches and the like. In general, modified starches are products prepared by chemical treatment of starches, for example, acid treatment starches, enzyme treatment starches, oxidized starches, cross-bonding starches, and other starch derivatives

The buffer and/or an alkalizing agent may be, but is not limited to, ammonium hydroxide, sodium hydroxide, sodium carbonate, sodium citrate, trisodium phosphate and/or disodium phosphate. In one embodiment, the buffer is disodium phosphate.

In some embodiments of the softgel capsule, the fill material may include at least one active pharmaceutical ingredient.

The fill composition of the softgel composition may further include additional ingredients. Those additional ingredients may be flavoring agents, sweetening agents, coloring agents, fillers, an antioxidant, synthetic dyes, mineral oxides, water, acrylates copolymer, xanthan gum, glycerin, butylene glycol, aminomethyl propanol, aloe barbadensis leaf extract, active ingredient, a lipid, or a combination thereof.

In some embodiments, the lipid may be selected, without limitations, from the group consisting of, almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, animal or vegetable fats, e.g., in their hydrogenated form, free fatty acids and mono-, di-, and tri-glycerides with C8-, C10-, C12-, C14-, C16-, C18-, C20- and C22-fatty acids, and combinations thereof.

In some embodiments, the antioxidant may be butylated hydroxytoluene (BHT), butylated hydroxyanisole (BHA), or a combination thereof.

In certain embodiments of the invention, the softgel may be spherical, oval, oblong or twist-off.

In another embodiment of the present disclosure, a method for marking a softgel capsule is provided. The method may include irradiating a softgel capsule with a laser, wherein the softgel capsule includes a shell composition and a fill material. The shell composition may include a marking component.

In some embodiments of the method, the irradiating may be performed using a wavelength below about 400 nm. In another embodiment of the method, the irradiating may be performed using a wavelength of about 100 nm, about 150 nm to about 400 nm, about 200 nm to about 350 nm, or about 250 nm to about 300 nm.

In some embodiments of the method, the irradiating may be performed using a UV laser.

In some embodiments, the irradiating may be performed by pulsing a laser towards the softgel capsule. In some embodiments, the laser may be pulsed for less than about 15 nsec. In some embodiments, the laser may be pulsed for about 1 nsec, about 2 nsec, about 5 nsec, about 7 nsec, about 10 nsec, about 12 nsec, or about 15 nsec.

In an embodiment of the method, after irradiating the softgel capsule, the marking component in the shell composition may undergo a color change. The color change may include a darkening of the marking component in the shell composition. In some embodiments, the color change may include forming black pigmentation in the shell composition to form a marking.

In embodiment of the method, the laser may have a power of about 10 watts to about 30 watts, about 15 to about 25 watts, or about 17.5 watts to about 22.5 watts.

In some embodiments of the method, the marking component may include a metal oxide. In certain embodiments, the metal oxide may include an iron oxide. The iron oxide may be an iron oxide red, iron oxide yellow, iron oxide black, iron oxide brown, or a combination thereof. In certain embodiments, the marking component does not include titanium dioxide. In other embodiments of the softgel capsule, the marking component is substantially free of titanium dioxide. Titanium dioxide has been believed to be required to achieve a color change in a shell of a softgel capsule, such as a gelatin shell. The present application has found that other metal oxides, such as iron oxide, can achieve similar color change without the use of titanium dioxide.

In some embodiments, the shell composition may further include a plasticizer and gelatin.

Without being limited to a theory, the inventors believe that the UV laser reacts with the marking component in the shell composition and causes a color change through the light of the UV laser. For example, it is believed that when the marking component is iron oxide, the iron oxide reacts with the UV laser to darken and form a mark in the shell composition. In some embodiments, the laser irradiation may be applied to the softgel capsule at a wavelength of about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, or about 400 nm, or any value therein.

In another embodiment, a marking formulation is provided. The marking formulation may include a marking component, a plasticizer and gelatin. In some embodiments, laser irradiation may be applied to the marking formulation at a wavelength of about 100 nm to about 400 nm, causing the marking component to change color.

In some embodiments, the marking component may include a metal oxide. In certain embodiments, the metal oxide may include an iron oxide. The iron oxide may be an iron oxide red, iron oxide yellow, iron oxide black, iron oxide brown, or a combination thereof. In certain embodiments, the marking component does not include titanium dioxide. In other embodiments of the softgel capsule, the marking component is substantially free of titanium dioxide.

In an embodiment, the marking component may be included in the marking formulation in an amount of 0.001 wt % to about 10 wt %, about 0.005 wt % to about 9 wt %, about 0.01 wt % to about 8 wt %, about 0.05 wt % to about 7 wt %, about 0.1 wt % to about 6 wt %, about 0.5 wt % to about 5 wt %, about 1 wt % to about 4 wt %, about 1.5 wt % to about 3 wt %, or about 2 wt % to about 2.5 wt %, based on a total weight of the marking formulation. In some embodiments, the marking component may be included in an amount of about 0.001 wt %, about 0.002 wt %, about 0.005 wt %, about 0.01 wt %, about 0.05 wt %, about 0.1 wt %, about 0.25 wt %, about 0.5 wt %, about 0.75 wt %, about 1 wt %, about 1.5 wt %, about 2 wt %, about 2.5 wt %, about 3 wt %, about 4 wt %, about 5 wt %, about 6 wt %, about 7 wt %, about 8 wt %, about 9 wt %, or about 10 wt %, or any value therein, based on the total weight of the marking formulation.

In some embodiments, the metal oxide may be included in an amount of about 1 gram to about 5 grams, about 1.2 grams to about 4.5 grams, about 1.4 grams to about 4 grams, about 1.6 grams to about 3.5 grams, about 1.8 grams to about 3 grams, or about 2 grams to about 2.5 grams, based on 100 grams of the marking formulation. In other embodiments, the metal oxide may be included in the marking formulation in an amount of about 1 gram, about 1.2 grams, about 1.4 grams, about 1.6 grams, about 1.8 grams, about 2 grams, about 2.5 grams, about 3 grams, about 3.5 grams, about 4 grams, about 4.5 grams, or about 5 grams, based on 100 grams of the marking formulation.

In some embodiments, the plasticizer may be included in the marking formulation in an amount of about 5 wt % to about 40 wt %, about 10 wt % to about 35 wt %, about 15 wt % to about 30 wt %, or about 20 wt % to about 25 wt %, based on a total weight of the marking formulation. In some embodiments, the plasticizer may be included in an amount of about 5 wt %, about 10 wt %, about 15 wt %, about 20 wt %, about 25 wt %, about 30 wt %, about 35 wt %, or about 40 wt %, or any value therein.

In some embodiments, the gelatin may be included in the marking formulation in an amount of about 20 wt % to about 70 wt %, about 25 wt % to about 65 wt %, about 30 wt % to about 60 wt %, about 35 wt % to about 55 wt %, or about 40 wt % to about 50 wt %, based on a total weight of the marking formulation. In some embodiments, the gelatin may be included in an amount of about 20 wt %, about 25 wt %, about 30 wt %, about 35 wt %, about 40 wt %, about 45 wt %, about 50 wt %, about 55 wt %, about 60 wt %, about 65 wt %, or about 70 wt %, based on total weight of the marking formulation.

In some embodiments of the marking formulation, the gelatin may be Type A gelatin, Type B gelatin, a hide gelatin, a bone gelatin, or a combination thereof. In some embodiments, the gelatin may include 150 bloom gelatin, 200 bloom gelatin, 250 bloom gelatin, or a combination thereof.

In some embodiments of the marking formulation, the plasticizer may include glycerol, glycerin, sorbitol, sorbitol sorbitan solution, triacetin, polysorbate, or combinations thereof or any of the plasticizers described herein. In some embodiments, the plasticizer may be glycerol.

In some embodiments, the marking formulation does not include titanium dioxide. In other embodiments, the marking formulation is substantially free of titanium dioxide. As noted above, it was found that color change may be achieved with the use of a different metal oxide, such as iron oxide, without the use of titanium dioxide or titanium oxide.

In some embodiments, the marking formulation may include water.

In an embodiment of the marking formulation, laser irradiation may be applied to the marking formulation at a wavelength of about 100 nm to about 400 nm. In some embodiments, the laser irradiation may be applied using a UV laser.

Any pharmaceutically active ingredient may be used for purposes of the present invention. Suitable pharmaceutically active ingredients include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g., clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sesquioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic, ginger root, gingko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pranoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, fisalamine, fenbufen, fenoprofen, flurbiprofen, ibuprofen, indometacin, ketoprofen, ketorolac, lornoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobamate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilators (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2,4,6,-trioxohexahydropyrimidine). Barbiturates include, but are not limited to, amobarbital, aprobarbital, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparaginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

In certain embodiments, other solid oral dosage forms besides softgels, such as tablets or hard capsules, may include a marking formulation and be laser irradiated as described herein.

### Examples

Several sample softgel capsules were prepared using a variety of marking formulations which were tested using a UV laser. In each example capsule, glycerol, purified water, and gelatin were combined with a marking formulation according to Table 1.

TABLE 1
Marking Formulation of Capsule

| Example | Metal oxide | Amount of Metal oxide (g) |
|---|---|---|
| Sample 1 | Iron Oxide Red | 0.8 |
| | Iron Oxide Yellow | 1.2 |
| Sample 2 | Iron Oxide Red | 0.05 |
| | Iron Oxide Yellow | 2.34 |
| Sample 3 | Iron Oxide Red | 0.2 |
| | Iron Oxide Yellow | 2.1 |
| Sample 4 | Iron Oxide Yellow | 2.4 |

The capsules of Samples 1-4 were tested to see if a reaction when applying a laser irradiation to the capsules at a wavelength of about 100 nm to about 400 nm. Each of the samples were prepared with at least one iron oxide and did not include titanium dioxide. A UV laser with a power of 15 watts was used for the marking. It was found that all formulations without titanium dioxide and with iron oxide reacted with the UV laser. That is, a color change was achieved. To verify this, a placebo capsule was prepared including natural gelatin and only red iron oxide and purified water in the shell. A UV laser with a power of 15 watts was applied. The inventors found that a color change was achieved when marking with a UV laser as can be seen in the FIGURE.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

1. A softgel capsule comprising:
a fill material; and
a shell composition, wherein the shell composition comprises a marking formulation including a marking component comprising iron oxide,
wherein laser irradiation is applied to the softgel capsule at a wavelength of about 100 nm to about 400 nm, causing the marking component to change color.

2. The softgel capsule of claim 1, wherein the marking component further comprises an additional metal oxide.

3. The softgel capsule of claim 1, wherein the marking formulation further comprises a plasticizer and gelatin.

4. The softgel capsule of claim 1, wherein the iron oxide is iron oxide red, iron oxide yellow, iron oxide brown, iron oxide black, or combination thereof.

5. The softgel capsule of claim 1, wherein the marking formulation is substantially free of titanium dioxide.

6. The softgel capsule of claim 1, wherein the marking formulation does not include titanium dioxide.

7. (canceled)

8. The softgel capsule of claim 1, wherein the laser irradiation is achieved using a UV laser.

9. A method for marking a softgel capsule comprising:
irradiating a softgel capsule with a laser, wherein the softgel capsule includes a shell composition and a fill material, and wherein the shell composition includes a marking formulation having a marking component comprising iron oxide.

10. The method of claim 9, wherein the irradiating is performed using a wavelength below about 400 nm.

11. (canceled)

12. The method of claim 9, wherein the irradiating is performed using a UV laser.

13. The method of claim 9, wherein after the irradiating of the softgel capsule, the marking component undergoes a color change.

14. (canceled)

15. (canceled)

16. The method of claim 1, wherein the marking formulation is substantially free of titanium dioxide.

17. The method of claim 1, wherein the marking formulation does not contain titanium dioxide.

18. A marking formulation comprising:
a marking component comprising iron oxide;
a plasticizer; and
gelatin,
wherein laser irradiation is applied to the marking formulation at a wavelength of about 200 nm to about 400 nm, causing the marking component to change color.

19. (canceled)

20. (canceled)

21. The marking formulation of claim 18, wherein the marking component is included in an amount of about 0.001 wt % to about 10 wt % based on a total weight of the marking formulation.

22. The marking formulation of claim 18, wherein the iron oxide is included in an amount of about 1 gram to about 5 grams based on 100 grams of total weight of the marking formulation.

23.-28. (canceled)

29. The marking formulation of claim 18, wherein the marking formulation is substantially free of titanium dioxide.

30. The marking formulation of claim 18, wherein the marking formulation does not comprise titanium dioxide.

31. The marking formulation of claim 18, further comprising water.

32. The marking formulation of claim 18, wherein the laser irradiation is applied using a UV laser.
